(19) United States
(12) Patent Application Publication
(10) Pub. No.: US 2026/0322397 A1
(43) Pub. Date: Oct. 15, 2026

(54) COMPRESSION OF TIME SERIES MEDICAL DATA

(71) Applicant: KONINKLIJKE PHILIPS N.V., EINDHOVEN (NL)

(72) Inventors: SYRUS CSABA NEMAT-NASSER, San Diego, CA (US); JOSHUA MICHAEL BURNS, San Diego, CA (US)

(21) Appl. No.: 19/293,150

(22) Filed: Aug. 7, 2025

Related U.S. Application Data
(60) Provisional application No. 63/687,937, filed on Aug. 28, 2024.

(51) Int. Cl. H03M 7/30; G16H 40/67

(52) U.S. Cl. CPC H03M 7/3064; G16H 40/67; H03M 7/6011; H03M 7/70

(57) ABSTRACT

An embodiment includes a method of time series data compression, including: obtaining, using a set of one or more processors, the time series data; applying, using the set of one or more processors, a first compression type to the time series data; identifying, using the set of one or more processors, a first set of segments of the time series data for which the first compression type produces an artifact in a residual signal; and indicating, using the set of one or more processors, the first set of segments. In an embodiment, a second compression type is applied to the first set of segments of the time series data, such that a final set of compressed data comprises the first set of segments compressed using lossless compression intermixed with the remaining segments compressed with lossy compression.

### CROSS-REFERENCE TO RELATED APPLICATIONS

This patent application claims the priority benefit under 35 U.S.C. § 119(e) of U.S. Provisional Application No. 63/687,937, filed on Aug. 28, 2024, the contents of which are herein incorporated by reference.

### BACKGROUND

### 1. Field

The disclosed subject matter generally pertains to data compression. Certain disclosed subject matter relates to compression techniques applied to biomedical data in a time series collected in an ambulatory monitoring context.

### 2. Description of the Related Art

Biomedical data is collected in a variety of settings, both in-hospital and during outpatient care. By way of example, mobile cardiac telemetry systems gather electrocardiogram (ECG) data in ambulatory settings (e.g., in the home environment or on-the-go). An example of mobile cardiac telemetry is use of an ambulatory patient sensor placed in a patch on the chest. The ambulatory patient sensor communicates wirelessly with a personal device, for example a smartphone having a dedicated mobile application (“app”), which is in turn connected (via the mobile phone network) to a remote system, such as an analysis server. An example of a mobile cardiac telemetry system is the Philips BioTel mobile cardiac outpatient telemetry (MCOT) system.

### SUMMARY

For biomedical time series data, compression provides savings in terms of storage and transmission. In the hospital setting, it may be desirable to use only lossless compression techniques because transmission latency is not a factor; however, such an approach does not offer maximal data size reduction for data storage, such as archival data storage. For time sensitive contexts, such as ambulatory monitoring, time latency is to be addressed. While high compression is needed, it may also be desirable to ensure that the clinically relevant features of the signal are preserved. This balance between preservation of clinically relevant features and high data compression can be challenging in the ambulatory monitoring context where motion artifacts and other sources of noise are common. Conventional approaches to artifacts and noise include destructive filtering as a preprocessing step, which is sub-optimal given that potentially clinically relevant data is irreversibly removed.

An embodiment therefore provides a piecewise data compression process that overcomes the limitations of conventional data compression techniques by identifying artifacts impacting compression in a first phase and allowing for segmenting of the signal on the basis thereof, prior to final compression and transmission of a complete compressed data set.

In summary, an embodiment provides a method of time series data compression, comprising: obtaining, using a set of one or more processors, the time series data; applying, using the set of one or more processors, a first compression type to the time series data; identifying, using the set of one or more processors, a first set of segments of the time series data for which the first compression type produces an artifact in a residual signal; and indicating, using the set of one or more processors, the first set of segments.

In an embodiment, the method comprises applying a second compression type to the first set of segments of the time series data. In an embodiment, the first compression type is lossy and the second compression type is lossless.

In an embodiment, the method comprises combining the first set of segments with remaining segments to form a final set of compressed data for the time series data. In an embodiment, the final set of compressed data comprises the first set of segments compressed using lossless compression intermixed with the remaining segments compressed with lossy compression.

In an embodiment, the method comprises combining the first set of segments with remaining segments to form a final set of compressed data for the time series data, wherein the set of one or more processors operate on a mobile biomedical data collection device; and transmitting the final set from the mobile biomedical data collection device to a remote device. In an embodiment, the time series data comprises ambulatory biomedical data. In an embodiment, the artifact results from one or more of motion of, or patient connectivity to, the mobile biomedical data collection device. In an embodiment, the artifact comprises one or more of a motion artifact and a baseline wander artifact. In an embodiment, the mobile biomedical data collection device comprises a cardiac monitoring patch.

In an embodiment, the method comprises combining the first set of segments with remaining segments to form a final set of compressed data for the time series data; and storing the final set in a storage device.

In an embodiment, the artifact in the residual signal is identified using a threshold. In an embodiment, the identifying comprises: generating the residual signal based on compression output produced by the applying the first compression type to the time series data; and comparing the residual signal to the threshold. In an embodiment, the threshold comprises a deviation value for the residual signal as compared to the time series data. In an embodiment, the deviation value is a fraction of a maximum magnitude of a respective signal amplitude of the time series data.

An embodiment provides a system comprising components such as a mobile biomedical data collection device. In an embodiment, the system comprises a set of one or more processors and executable code stored in a non-transitory storage medium, the executable code being used by the set of one or more processors to perform one or more of the methods, or part thereof, as described herein.

An embodiment provides a computer program product comprising a non-transitory computer readable medium comprising code executable by a set of one or more processors to perform one or more of the methods, or part thereof, as described herein.

The foregoing is a summary and thus may contain simplifications, generalizations, and omissions of detail; consequently, those skilled in the art will appreciate that the summary is illustrative only and is not intended to be in any way limiting.

These and other features and characteristics of the example embodiments, as well as the methods of operation and functions of the related elements of structure and the combination thereof, will become more apparent upon consideration of the following description and the appended claims with reference to the accompanying drawings, all of which form a part of this specification. It is to be expressly understood, however, that the drawings are for the purpose of illustration and description only and are not intended as a definition of the limits of a claimed invention.

### BRIEF DESCRIPTION OF THE DRAWINGS

FIG. 1 illustrates an example system according to an embodiment.

FIG. 2 illustrates an example method according to an embodiment.

FIG. 3 illustrates an example method according to an embodiment.

FIG. 4 illustrates a diagram of example system components according to an embodiment.

### DETAILED DESCRIPTION OF EXAMPLE EMBODIMENTS

The described features, structures, or characteristics of the example embodiments may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, etc. In other instances, well known structures, materials, or operations are not shown or described in detail to avoid obfuscation.

Current data compression techniques provide some savings in terms of reducing data size for storage and transmission of biomedical time series data. In the hospital setting, lossless compression may be used because transmission latency is not a factor; however, this results in sub-optimal data compression, which may be relevant for certain contexts such as archival storage. For ambulatory monitoring, however, time latency should be addressed as in the case of remote ECG monitoring, such as provided by a mobile biomedical data collection device in the form of a cardiac monitoring patch.

In the ambulatory context, data may be transmitted to a remote device, such as a cloud server running a machine learning service that identifies clinically relevant features, to provide near real-time indicating or alerting for clinicians. For example, deep learning techniques are used for analyzing remotely collected ECG data that is uploaded to the cloud. These deep learning techniques are accurate but are not implemented on mobile biomedical data collection devices because they consume high memory and compute, which may not be available locally and, even if available, would significantly impact battery life.

While high compression is needed in certain contexts, such as for ambulatory monitoring, it is also desirable to ensure that the clinically relevant features of the signal are preserved, such as, for ECG data, wave complexes such as QRS complex and related features. This preservation through compression and decompression (to produce a reconstructed signal) allows for viewing and analysis by clinicians, automated processes, or a combination thereof. The preservation of clinically relevant features for reproduction in a reconstructed signal can be challenging in certain time series data due to artifacts contained in the time series data. For example, ambulatory monitoring data commonly contains motion artifacts and other sources of noise. This occurs in ambulatory data due to a variety of circumstances such as patient connectivity being less than optimal (including physical connectivity issues or misplacement of a mobile monitoring device) as well as the natural motion of a monitoring device when attached to a patient.

One conventional approach for time-sensitive compression of ambulatory monitoring data is to have local ECG identification processes run on the mobile biomedical data collection device. These are used to identify limited events that are compressed and sent to the cloud over wireless networks, often cellular networks. To manage cost and latency, the data sent is not a full disclosure record, e.g., sending samples of ECG data that have been identified as clinically relevant events. This approach has the disadvantage that the full disclosure record ECG data is not available, only the events that have been sent. Otherwise, a full disclosure record may be reported but with significant latency, e.g., after a device is connected to Wi-Fi. The disadvantage of this latency is the time delay to get the data and related analysis results, making it insufficient for patients that may be on the verge of an emergent medical condition. In addition, techniques that use high compute or memory may not be viable on a local client device due to resources and/or consumption of battery life.

Accordingly, an embodiment provides a hybrid compression technique where a lossy compression is applied to time series data to identify segments or areas in the data that are not well managed, for example the lossy compression applied is not well tuned in terms of preserving relevant features. For such segments, a second phase of compression is applied, using a second compression type. For example, lossless compression is applied to the segments to preserve any features that may be relevant. An embodiment may be used to produce a final compressed data set, intermixing the lossy and lossless compressed segments, aligned such that a reconstructed signal, with different applications of compression techniques, is produced with negligible effect on the reconstructed signal, e.g., to a human reviewer or automated analysis process. For example, the data of the reconstructed signal may be aligned for continuity on the basis of a shared feature between segments subjected to different types of compression techniques, for example a baseline signal used to scale each segment of the reconstructed signal.

An embodiment applies an approach that reduces network and processing cost as well as latency throughout the entire pipeline, from local capture and storage through clinical validation by technicians and physicians. An embodiment also reduces the size or amount of data that needs to be stored, for example in comparison with lossless compression techniques used in certain storage contexts such as in-hospital. While certain non-limiting examples are provided herein with respect to ECG data, an embodiment may be used to apply compression to various other data types, such as, without limitation, electroencephalogram (EEG) data and electromyography (EMG) data.

The description now turns to the figures. The illustrated example embodiments will be best understood by reference to the figures. The following description is intended only by way of example, and simply illustrates certain example embodiments.

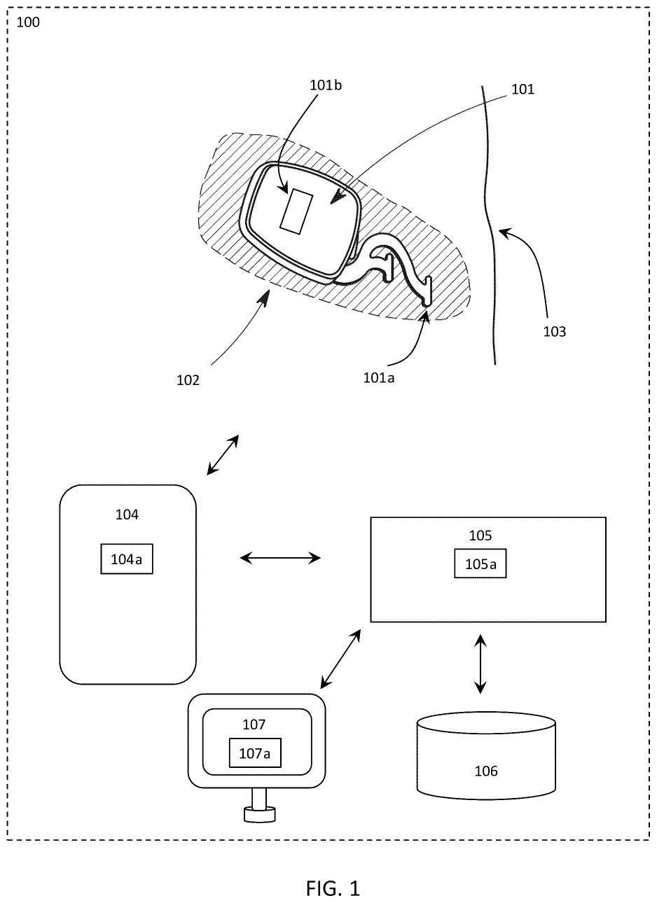

Illustrated in FIG. 1 is an example system 100 for mobile cardiac telemetry. System 100 includes a mobile biomedical data collection device 101 that is adhered using a patch 102, e.g., adhesive, to a patient 103. Mobile biomedical data collection device 101 in turn may include a variety of sensing devices, by way of example and not limitation, ECG sensor leads or electrodes 101a and a motion or an activity sensor 101b such as an accelerometer for detecting movement of mobile biomedical data collection device 101 (and thus patient 103). Mobile biomedical data collection device 101 may monitor, sense and report other continuous patient metrics, for example heart rate sensed by a heart rate sensor, blood pressure sensed by a blood pressure sensor, or similar. As such, mobile biomedical data collection device 101 is configured to detect a continuous patient metric such as ECG data, heart rate, blood pressure or the like, and may obtain and report a time series of biomedical data. In an embodiment, more than one mobile biomedical data collection device 101 may be utilized, or certain functionality of mobile biomedical data collection device 101 may be provided by a distributed system, e.g., separate sensor devices cooperating to sense and report a continuous patient metric as time series data. Further, in an embodiment other data types may be collected, for example time series data of other sensors such as EEG data, etc.

In an embodiment, system 100 also comprises a local device 104 such as a personal user device, e.g., a smartphone or the like, with a module 104a configured to communicate, e.g., wirelessly, with mobile biomedical data collection device 101. Local device 104 may also communicate, e.g., wirelessly, with a remote device 105, e.g., a server of a clinical system, which in turn operates a module 105a configured to communicate with a database 106 and display 107, such as a clinician display, which operates module 107a configured to communicate to one or more of local device 104 and remote device 105. In an embodiment, modules 104a, 105a, and 107a provided in local device 104, remote device 105, and display 107, respectively, may comprise computer code, software or firmware, such as applications, as well as a set of one or more processors, configured execute the code to process biomedical data such as time series data for a continuous patient metric e.g., for analysis of clinical features in a could device or remote device 105, or storage in database 106. It will be understood that system components may be combined as well as be distributed in system 100 variously.

In an embodiment, biomedical data may be recorded and reported by mobile biomedical data collection device 101 in the form of time series data, for example transmitted to local device 104 and, directly or indirectly, to remote device 105 for further analysis such as application of a machine learning classification process to identify clinically relevant features to trigger one or more workflows, for example sending a notification to a clinician of an emergent cardiac event.

As described herein, an embodiment may apply data compression to the time series data prior to transmission or storage. By way of example, mobile biomedical data collection device 101 or local device 104 may apply data compression, using a set of one or more processors, prior to transmission of the time series data. In an embodiment, mobile biomedical collection device 101 includes program instructions stored in a non-transitory memory that are executed by the set of one or more processors to compress the biomedical time series data prior to transmission or persistent storage thereof. For example, mobile biomedical collection device 101 compresses data prior to transmitting it to remote device 105.

For ambulatory monitoring, ECG compression enables mobile biomedical data collection device 101 to provide full disclosure or complete time series data reporting to remote device 105 for accurate and complete analysis while maintaining the near real-time latency. This improves cost with respect to both time and network bandwidth usage across data storage, time of transmission over wireless networks including Bluetooth, cellular, and Wi-Fi, cost of transmission over cellular networks to reach remote device 105, reducing latency of delivery to cloud analysis processes and ECG clinicians (technicians, physicians, etc.).

The ambulatory environment for biomedical data such as ECG data is challenged by the mobility of the patient and the limitations of mobile biomedical data collection device 101. For example, mobile biomedical collection device may be limited to 1 or 2 leads. Further, in many cases patients are asked to perform self-application of mobile biomedical data collection device 101. Motion artifacts are common, for example when the patient moves or bumps mobile biomedical data collection device 101. These artifact producing events happen more often than in a non-ambulatory hospital setting. One way to remove such artifacts or noise is digital filtering, such as applying a zero-phase high-pass filter. Such digital filtering, however, has multiple disadvantages. For example, applying a cutoff frequency, e.g., greater than 0.05 Hz, causes the ECG signal to lose its certification for ST Segment analysis, and applying a filter is not reversible such that the human reviewer or automated analysis process may lose useful context for clinical interpretation.

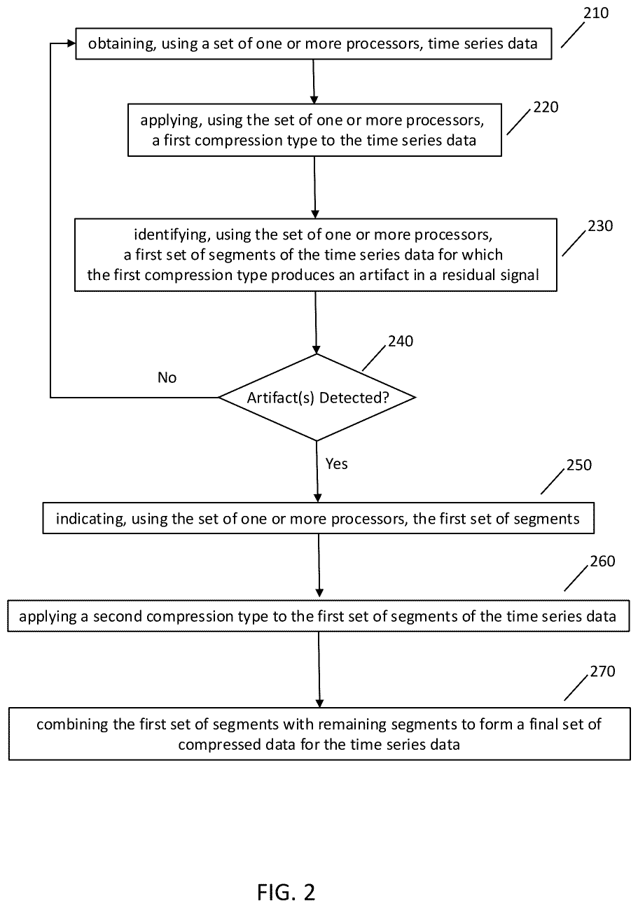

Referring to FIG. 2, a compression method of an embodiment allows for high compression ratios, e.g., 10× or higher, for signals that include artifacts such as baseline wander and motion artifacts. An embodiment does not require that these artifacts be removed from the signal prior to applying the data compression and so avoids any destructive filtering or alteration of the time series data. Instead, the presence of such artifacts is leveraged as information that is reportable and actionable, for example in making a choice to apply a different compression technique, such as application of a lossless compression technique to artifact containing segments.

Illustrated in FIG. 2, in an embodiment a method includes obtaining, using a set of one or more processors, the time series data at 210. For example, mobile biomedical data collection device 101 may obtain ECG data using leads 101a. By way of specific example, mobile biomedical data collection device 101 may collect ECG time series data using a single channel as a 12-bit signed waveform sampled at 256 Hz. As another specific example, mobile biomedical data collection device 101 may obtain a 2-channel, 12-bit signed waveforms sampled at 250 Hz. The duration of time series data obtained at 210, e.g., an amount of ECG signal data obtained for compression at one time, may be empirically determined. For example, the amount of data obtained may be based on the average compression ratio with consideration for the latency between collection and analysis context. An example duration of time series data for mobile biomedical data collection device 101 that is configured to collect single or multi-channel ECG data is on the order of 2-3 minutes. This amount of data may be tuned for various different data or sensor types and is subject to further processing in terms of applying a data compression technique, as further described herein.

At 220, a method according to an embodiment includes applying, using the set of one or more processors, a first compression type to the time series data obtained at 210. For example, a lossy compression process may be applied by mobile biomedical data collection device 101 to the time series data at 220. As a non-limiting example, a lossy compression technique that is tuned to the type of data (e.g., ECG data) and signal features sought to be preserved (e.g., QRS waveforms and related features) may be used. As a specific example, a lossy compression technique based on a discrete wavelet transform may be used to compress an ECG waveform with compression ratios of 10× or higher. Under nominal conditions, this type of lossy compression technique preserves the clinically relevant features of the ECG signal at very high compression ratios, but such lossy compression techniques are known to fail to preserve these features in the presence of artifacts such as motion artifacts or other noise such as baseline wander. Thus, an artifact may be produced in segments of the signal that do not compress well in terms of forming a signal similar to the original time series data using the lossy compression applied at 220. In an embodiment, compression applied at 220 may take other forms, for example a lossy compression that uses a machine learning process, for example implemented using a deep learning autoencoder architecture where the layer in the middle of the trained autoencoder has a sparse, quantized activation supporting an optimal compression ratio for the time series data type. In some embodiments, a lossy compression is developed using a large set of examples of the biomedical time series data type, where these examples are used as training data for the deep learning autoencoder. Such lossy compression type examples are not limiting but rather are provided to illustrate that any effective method to develop a lossy compression that preserves relevant clinical features may be employed.

As shown in FIG. 2, an embodiment identifies, using the set of one or more processors, a first set of segments of the time series data for which the first compression type produces an artifact in a residual signal at 230. An artifact in the residual signal occurs because the original signal is outside the range of biomedical signals for which the lossy compression is optimized. The definition of an artifact accommodates various types of features of interest for retaining in the signal, lossy compression tuning to such features of interest, and data types. There are various approaches that may be used to implement identification of an artifact at 230, ranging from manual review of the original and residual signals through programmatic routines, including for example application of a dynamic or predetermined threshold, using a machine learning classification model (trained with human review and validation during a training phase), or the like. By way of example, in a residual signal generated after application of lossy compression, an embodiment identifies at 230 deviations of segments (time durations) of the residual signal from a noise baseline that are above a dynamic threshold. In an embodiment, the dynamic threshold is a fixed fraction of the maximum magnitude of the signal amplitude of the original time series data. For ECG signals this approach works well and a human reviewer can notice spikes in the residual signal indicative of artifacts, which may be manually indicated or distinguished. A manual process may be used, for example, to form a training data set for training an automated classification technique, such as a machine learning model that classifies artifact and non-artifact segments. In other embodiments, a different method may be used to identify artifacts and associate these with segments of the time series data, e.g., application of a combination of thresholding and automated classification.

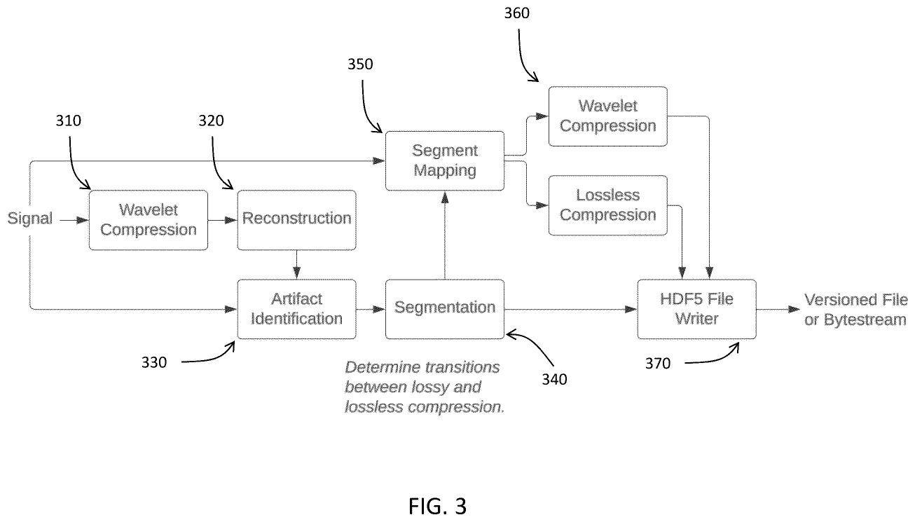

Having identified an artifact in the time series per the applicable definition for a given type of data, an embodiment identifies an associated segment of the time series for the artifact at 230. For example, an embodiment may determine a time range delineated by or associated with the artifact to identify a segment. Thus, a set of one or more segments are identified at 230 for the time series data obtained at 210. An embodiment may determine a segmentation of the time series into alternating artifact and non-artifact intervals, where the signal starts with one or the other of these. If no such artifacts are detected, as indicated at 240, the lossy compression may be applied to the next set of data obtained, the entire time series, etc. However, each identified segment at 230 is indicated at 250 by an embodiment, which facilitates differential handling of such segments. In an embodiment, the segments may be indicated using metadata descriptive of the segment type, for example as packaged into a file for transmission as described in connection with FIG. 3.

As illustrated at 260 of FIG. 2, identified segments may be subjected to a different type of compression, for example lossless compression, as further described herein. The indication provided by an embodiment at 250 may be used for other purposes, for example noting areas of the time series via addition of metadata associated therewith, which may facilitate providing visual indications associated with the residual or reconstructed signal display, markers for excision or removal of the segments according to a storage workflow, etc.

Following the non-limiting example of handling ambulatory biometric data, an embodiment may compress each segment using a different compression technique. Segments indicated or marked as containing an artifact at 250 are compressed using lossless compression at 260, whereas segments indicated or marked as non-artifact containing are compressed using a lossy compression technique. An embodiment therefore allows for a combination of the first set of segments with remaining segments to form a final set of compressed data, or full disclosure set, for the time series data as indicated at 270. As such, in an embodiment a final set of compressed data comprises the first set of segments compressed using lossless compression intermixed with the remaining segments compressed with lossy compression. This final set of compressed data may be transmitted, for example from mobile biomedical data collection device 101 to remote device 105 for application of machine learning classification, triggering a workflow depending upon the classification result. For example, remote device 105 may decode the segments with a decoder appropriate for the indicated compression type used per segment and classify a portion or segment of a signal as indicative of a clinical feature, e.g., atrial fibrillation in the case of ECG data. Such classification output may be used in downstream tasks, such as triggering an alerting workflow, sending a message to a clinician for appropriate review and follow up, visually updating a display of the reconstructed signal to indicate segments and related feature classifications, etc.

With continued reference to FIG. 2, combining at 270 may include encoding the compressed segments into a predetermined format. In some embodiments, this format is a Hierarchical Data Format version 5 (HDF5) file that supports inclusion of multiple named arrays of typed data. For example, an embodiment may provide a compact description of the segmentation, e.g. ‘LWLW’ indicates four segments starting with an artifact segment using lossless compression (L) followed by a lossy compression (W for wavelet type compression), etc. From the file's compact description, each segment is accessed from named elements computed automatically, e.g. ‘L0’ is the root name of the first lossless segment, ‘W1’ is the root name for the data elements of the first wavelet compressed segment, which follows ‘L0’ in the reconstruction, and so on. In connection with decompression, the signal segments may be combined and aligned to reproduce the ECG signal with continuity between the segments having been compressed using different techniques and recovered.

FIG. 3 illustrates an example of functional modules and relations there-between for an example time series data compression process performed prior to storage or transmission of the time series data, e.g., performed by mobile biomedical data collection device 101 or local device 104 of FIG. 1. As shown in FIG. 3, a signal comprising time series data is obtained and subject to lossy compression, e.g., wavelet compression as indicated at 310. This compressed signal is then decompressed to generate a reconstructed signal as indicated at 320. The reconstructed signal is compared to the original signal and subjected to a process for artifact identification at 330, e.g., comparison of a residual signal to a baseline threshold determined from analysis of the original time series data, where the residual is defined as the difference between the original and the reconstructed time series data.

As described herein, the identification of artifact(s) at 330 permits segments of the signal to be indicated, for example delineating portions having artifact(s) from those where artifacts are absent, such as delineating transition areas in the signal. In an embodiment, the segmentation is used to mark or indicate segments, which may take the form of a segment mapping as indicated at 350, e.g., metadata delineating the artifact and non-artifact containing segments or portions of the signal.

In an embodiment, the segment mapping at 350 may be used in a second phase of compression, which at least includes applying a different, second type of compression to the artifact containing segments. In the example of FIG. 3, two types of compression are applied in a second compression phase to the original signal, collectively indicated by 360, i.e., lossless compression is applied to the original signal for artifact containing segments to preserve any and all features of the signal in such segments, whereas lossy compression is applied to the remaining segments, i.e., for which the lossy compression is well tuned in terms of feature preservation. As will be appreciated, this facilitates retention of all data for the lossless compressed data segments for later manual, human review or processing by a program such as a machine learning classification process. It is also noted that the remaining segments may be identified, extracted, and used for combination with the lossless compressed data instead of operating on the original signal to again perform the lossy compression. However, the second compression phase may be utilized to update or change the lossy compression applied, for example based on the signal compression indicating many artifacts or a particular artifact type, i.e., steps indicated in FIG. 3 may be performed again depending on the outcome of one or more of the steps.

After the compression types have been applied at 360 per the segment map, an embodiment may encode a file for transmission, e.g., to remote device 105, using an appropriate format such as formed using an HDF5 file writer or similar functional module, as indicated at 370. This provides versioned file byte stream data for consumption by a target entity, e.g., remote device 105 hosting machine learning processes or the like. As indicated, the HDF5 file writer 370 may utilize the segment information, such as determined by segmentation module 340 or via obtaining segment map 350 to determine transitions between lossy and lossless compression for compilation of the file parts or array, for example as described in connection with FIG. 2.

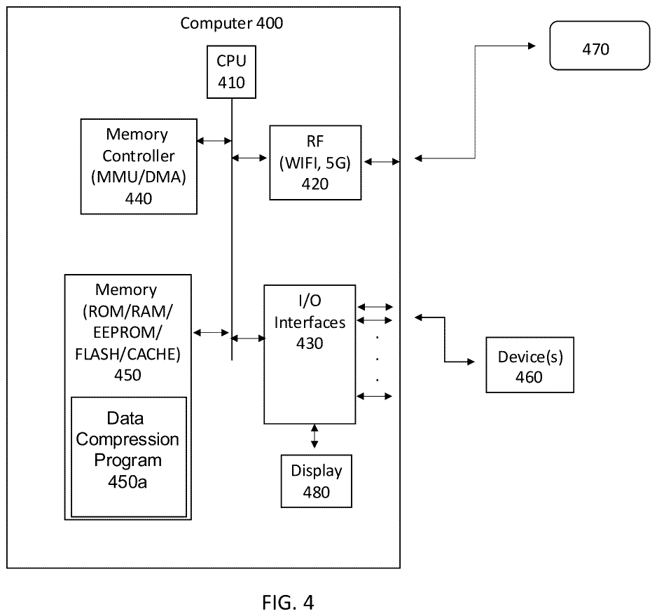

Referring to FIG. 4, it will be readily understood that certain embodiments can be implemented using any of a wide variety of devices or combinations of devices and components. In FIG. 4 an example of a computer 400 and its components are illustrated, which may be used in a device such as mobile biometric data collection device 101for implementing the functions or acts described herein, e.g., executing a data compression program 450a. Also, circuitry other than that illustrated in FIG. 4 may be utilized in one or more embodiments. The example of FIG. 4 includes certain functional blocks, as illustrated, which may be integrated onto a single semiconductor chip to meet specific application requirements.

One or more processing units are provided, which may include a central processing unit (CPU) 410, one or more graphics processing units (GPUs), and/or micro-processing units (MPUs), which include an arithmetic logic unit (ALU) that performs arithmetic and logic operations, instruction decoder that decodes instructions and provides information to a timing and control unit, as well as registers for temporary data storage. CPU 410 may comprise a single integrated circuit comprising several units, the design and arrangement of which vary according to the architecture chosen.

Computer 400 also includes a memory controller 440, e.g., comprising a direct memory access (DMA) controller to transfer data between memory 450 and hardware peripherals. Memory controller 440 includes a memory management unit (MMU) that functions to handle cache control, memory protection, and virtual memory. Computer 400 may include controllers for communication using various communication protocols (e.g., I²C, USB, etc.).

Memory 450 may include a variety of memory types, volatile and nonvolatile, e.g., read only memory (ROM), random access memory (RAM), electrically erasable programmable read only memory (EEPROM), Flash memory, and cache memory. Memory 450 may include embedded programs, code, and downloaded software, e.g., data compression program 450a that provides coded methods such as illustrated and described in connection with FIGS. 2-3 (or parts thereof). By way of example, and not limitation, memory 450 may also include an operating system, application programs, other program modules, code, and program data, which may be downloaded, updated, or modified via remote devices.

A system bus permits communication between various components of the computer 400. I/O interfaces 430 and radio frequency (RF) devices 420, e.g., Wi-Fi and telecommunication radios, may be included to permit computer 400 to send data to and receive data from remote devices using wireless mechanisms, noting that data exchange interfaces for wired data exchange may be utilized. Computer 400 may operate in a networked or distributed environment using logical connections to one or more other remote computers or devices 470, such as a database that stores received, compressed time series data or a remote server offering machine learning processing of received, compressed time series data. The logical connections may include a network, such local area network (LAN) or a wide area network (WAN) but may also include other networks/buses. For example, computer 400 may communicate data with and between device(s) 460, for example personal user device(s) that provide communication and data connectivity to ambulatory patient sensors, etc.

Computer 400 may therefore execute program instructions or code configured to obtain, store, and analyze patient medical data and perform other functionality of the embodiments, such as described in connection with FIGS. 2-3. A user can interface with (for example, enter commands and information) the computer 400 through input devices, which may be connected to I/O interfaces 430. A display 480 or other type of output device may be connected to or integrated with the computer 400, for example via an interface selected from I/O interfaces 430.

It should be noted that the various functions described herein may be implemented using instructions or code stored on a memory, e.g., memory 450, that are transmitted to and executed by a processor, e.g., CPU 410. Computer 400 includes one or more storage devices that persistently store programs and other data. A storage device, as used herein, is a non-transitory computer readable storage medium. Some examples of a non-transitory storage device or computer readable storage medium include, but are not limited to, storage integral to computer 400, such as memory 450, a hard disk or a solid-state drive, and removable storage, such as an optical disc or a memory stick.

Program code stored in a memory or storage device may be transmitted using any appropriate transmission medium, including but not limited to wireless, wireline, optical fiber cable, RF, or any suitable combination of the foregoing.

Program code for carrying out operations according to various embodiments may be written in any combination of one or more programming languages. The program code may execute entirely on a single device, partly on a single device, as a stand-alone software package, partly on single device and partly on another device, or entirely on the other device. In an embodiment, program code may be stored in a non-transitory medium and executed by a processor to implement functions or acts specified herein. In some cases, the devices referenced herein may be connected through any type of connection or network, including a local area network (LAN) or a wide area network (WAN), or the connection may be made through other devices (for example, through the Internet using an Internet Service Provider), through wireless connections using a mobile network, or through a hard wire connection, such as over a USB connection.

An embodiment may be implemented in a variety of devices, including user devices such as mobile biomedical data collection device 101 running a mobile application, e.g., program 450a. In one embodiment, referring to FIG. 1, the obtaining of patient data and employing data compression program 450a are performed locally on mobile biomedical collection device 101; however, some or all of these acts may also be implemented via other devices and modules, for example local device 104 and module 104a, or in the case of storage, by a remote device 105 using module 105a, where existing, stored data is compressed for archival purposes, by way of example. An embodiment may also be provided as a distributed system, where data is obtained by remote device 105 and functions and acts of a data visualization program are at least partially performed in remote device 105, e.g., by module 105a, e.g., for displaying time series data on display 107, which may be an interactive display, for example facilitated by module 107a.

Therefore, an embodiment may include an application program configured to execute computer program instructions, for example as outlined at least in part in FIGS. 2-3, which in combination with device hardware, permit compression of biomedical data as described herein.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” or “the” preceding an element does not exclude the presence of a plurality of such elements. The mere fact that certain elements are recited in mutually different dependent claims does not indicate that these elements cannot be used in combination. The word “about” or similar relative term as applied to numbers includes ordinary (conventional) rounding of the number with a fixed base such as 5 or 10.

It is worth noting that while specific blocks are used in the figures, and a particular ordering of blocks has been illustrated, these are non-limiting examples. In certain contexts, two or more blocks may be combined, a block may be split into two or more blocks, or certain blocks may be re-ordered or re-organized or omitted as appropriate, as the explicit illustrated examples are used only for descriptive purposes and are not to be construed as limiting.

As used herein, the statement that two or more parts or components are “coupled” shall mean that the parts are joined or operate together either directly or indirectly, e.g., through one or more intermediate parts or components, so long as a link occurs. As used herein, “operatively coupled” means that two or more elements are coupled to operate together or are in communication, unidirectional or bidirectional, with one another. As used herein, the term “number” shall mean one or an integer greater than one (i.e., a plurality). As used herein a “set” shall mean one or more.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. A method of time series data compression, comprising:
obtaining, using a set of one or more processors, the time series data;
applying, using the set of one or more processors, a first compression type to the time series data;
identifying, using the set of one or more processors, a first set of segments of the time series data for which the first compression type produces an artifact in a residual signal; and
indicating, using the set of one or more processors, the first set of segments.

2. The method of claim 1, comprising applying a second compression type to the first set of segments of the time series data.

3. The method of claim 2, wherein the first compression type is lossy and the second compression type is lossless.

4. The method of claim 3, comprising combining the first set of segments with remaining segments to form a final set of compressed data for the time series data.

5. The method of claim 4, wherein the final set of compressed data comprises the first set of segments compressed using lossless compression intermixed with the remaining segments compressed with lossy compression.

6. The method of claim 1, comprising:
combining the first set of segments with remaining segments to form a final set of compressed data for the time series data;
wherein the set of one or more processors operate on a mobile biomedical data collection device; and
transmitting the final set from the mobile biomedical data collection device to a remote device.

7. The method of claim 6, wherein the time series data comprises ambulatory biomedical data.

8. The method of claim 7, wherein the artifact results from one or more of motion of, or patient connectivity to, the mobile biomedical data collection device.

9. The method of claim 8, wherein the artifact comprises one or more of a motion artifact and a baseline wander artifact.

10. The method of claim 9, wherein the mobile biomedical data collection device comprises a cardiac monitoring patch.

11. The method of claim 1, comprising:
combining the first set of segments with remaining segments to form a final set of compressed data for the time series data; and
storing the final set in a storage device.

12. The method of claim 1, wherein the artifact in the residual signal is identified using a threshold.

13. The method of claim 12, wherein the identifying comprises:
generating the residual signal based on compression output produced by the applying the first compression type to the time series data; and
comparing the residual signal to the threshold.

14. The method of claim 13, wherein the threshold comprises a deviation value for the residual signal as compared to the time series data.

15. The method of claim 14, wherein the deviation value is a fraction of a maximum magnitude of a respective signal amplitude of the time series data.

16. A system for time series data compression, comprising:
a set of one or more processors; and
a non-transitory storage medium comprising code that is executable by the set of one or more processors, the code comprising:
code configured to obtain the time series data;
code configured to apply a first compression type to the time series data;
code configured to identify a first set of segments of the time series data for which the first compression type produces an artifact in a residual signal; and
code configured to indicate the first set of segments.

17. The system of claim 16, comprising code configured to apply a second compression type to the first set of segments of the time series data.

18. The system of claim 17, wherein the first compression type is lossy and the second compression type is lossless.

19. The system of claim 18, comprising code configured to combine the first set of segments.

20. A computer program product for time series data compression comprising a non-transitory storage medium having code executable by a set of one or more processors, the code comprising:
code configured to obtain the time series data;
code configured to apply a first compression type to the time series data;
code configured to identify a first set of segments of the time series data for which the first compression type produces an artifact in a residual signal; and
code configured to indicate the first set of segments.
